(19) United States
(12) Patent Application Publication
(10) Pub. No.: US 2024/0360416 A1
(43) Pub. Date: Oct. 31, 2024

(54) MICROFLUIDIC BIOMIMETIC FIBER FOR CULTURED MEAT PRODUCTION AND PREPARATION METHOD THEREFOR AND USE THEREOF

(71) Applicant: NANJING AGRICULTURAL UNIVERSITY, Jiangsu (CN)

(72) Inventors: Jie WANG, Jiangsu (CN); Guanghong ZHOU, Jiangsu (CN); Xi DING, Jiangsu (CN)

(73) Assignee: NANJING AGRICULTURAL UNIVERSITY, Jiangsu (CN)

(21) Appl. No.: 18/765,308

(22) Filed: Jul. 7, 2024

Related U.S. Application Data
(63) Continuation of application No. PCT/CN2023/104613, filed on Jun. 30, 2023.

(30) Foreign Application Priority Data
Jul. 4, 2022 (CN) 202210777912.4

(51) Int. Cl. C12N 5/077; A23L 13/00; D01F 1/10; D01F 8/18

(52) U.S. Cl. CPC C12N 5/0658; A23L 13/00; D01F 1/10; D01F 8/18; C12N 2501/115; C12N 2513/00; C12N 2533/54; C12N 2533/74; C12N 2537/10; D10B 2505/00

(57) ABSTRACT

A microfluidic biomimetic fiber for cultured meat production and a preparation method therefor and use thereof are provided. The microfluidic biomimetic fiber has a shell-core structure; and an outer shell of the microfluidic biomimetic fiber is formed by cross-linking a polymer solution with cell non-adhesion, and an inner core wrapped by the outer shell is a hydrogel solution mixed with a seed cell. The present invention respectively introduces an inner phase fluid and an outer phase fluid into inner and outer phase channels of a microfluidic device, enables the two phase fluids in the channels of the microfluidic device to form a stable laminar flow structure by adjusting the flow velocities of the inner and outer phases, and extrudes same from a device outlet to obtain the biomimetic fiber.

### CROSS-REFERENCE TO RELATED APPLICATION

This application is a continuation of international application of PCT application serial no. PCT/CN2023/104613 filed on Jun. 30, 2023, which claims the priority benefit of China application no. 202210777912.4, filed on Jul. 4, 2022. The entirety of each of the above mentioned patent applications is hereby incorporated by reference herein and made a part of this specification.

### BACKGROUND

### Technical Field

The present invention belongs to the field of cultured meat and particularly relates to a microfluidic biomimetic fiber for cultured meat production and a preparation method therefor and use thereof.

### Description of Related Art

Meat contains rich essential nutrients such as protein, vitamins and minerals, and has become an important component of diet pagoda of residents in various countries in the world. With the increase of the world population and the meat demand year by year, the contradiction between the meat production mode depending on the livestock breeding industry, and the ecological resources, public health safety, ethics and the like is increasingly prominent, and thus it is of great significance to develop a novel meat production technology capable of replacing the traditional livestock breeding.

Under the background, cultured meat stands out and is a meat obtained by in-vitro culture of related cells relying on tissue engineering and the self-healing regeneration capacity of animal muscle tissues. It is expected to solve the problem of meat supply in the future by the in-vitro culture mode. At present, the mode of producing cultured meat is generally to give an appropriate carrier to seed cells, induce the seed cells to proliferate and differentiate to be mature on the carriers, and finally harvest the cultured meat. Common carrier materials for producing the cultured meat mainly include an animal protein scaffold, a plant protein scaffold, an acellular plant scaffold, a blocky hydrogel and the like. Despite the fair progress of research and use, the scaffold materials still have shortcomings in cell adherence, migration and fusion efficiency of seed cells, and cannot accurately simulate a fibrous basic physiological structure of muscle fibers in natural skeletal muscle, such that the capability of the seed cells to differentiate on the scaffold to form a mature muscle tissue is limited, and the production efficiency is low. Microfluidics is a classical technology in the field of tissue engineering, can control a trace amount of fluids in micro-sized channels, and is considered as a powerful means for preparing fibrous carriers. However, the use of the microfluidic technology in cultured meat production has not been reported.

### SUMMARY

Objective of the present invention: aiming at the problems existing in the prior art, the present invention provides a microfluidic biomimetic fiber for cultured meat production. The microfluidic biomimetic fiber prepared by the present invention effectively solves the problems that the existing cultured meat production mode has a long period and a complicated process, and cannot accurately simulate the in-vivo growth environment of seed cells, the synthesis of related proteins is less, and the production efficiency of the cultured meat is low.

The present invention further provides a preparation method for and use of the microfluidic biomimetic fiber.

Technical solution: in order to realize the above-mentioned objective, the microfluidic biomimetic fiber of the present invention has a shell-core structure; and an outer shell of the microfluidic biomimetic fiber is formed by cross-linking a polymer solution with cell non-adhesion, and an inner core wrapped by the outer shell is a hydrogel solution mixed with a seed cell.

The polymer solution with cell non-adhesion includes but is not limited to any one or more of sodium alginate, chitosan, pectin, carrageenan, and gellan gum; and the concentration of the polymer solution with cell non-adhesion is 10-50 mg/mL.

The hydrogel solution contains 30%-70% by volume of a biomaterial, 0.01%-1% by volume of a cross-linking agent, and the balance of a basic culture medium containing a calcium salt and 5×10⁶-5×10⁸ seed cells/mL.

The seed cell source includes but is not limited to any one or more of a pig, cattle, sheep, a chicken, a duck, a rabbit, fish, a shrimp, and a crab.

Preferably, the seed cell is derived from any one or more of a pig, cattle, sheep, a chicken, and a duck.

The seed cell includes but is not limited to one or more of a muscle stem cell, a myoblast, a muscle satellite cell, a muscle precursor cell, a bone marrow-derived mesenchymal stem cell, an adipose-derived mesenchymal stem cell, an induced pluripotent stem cell, a cardiac muscle cell, an adipose stem cell, an adipose precursor cell, a bone marrow-derived adipose adult cell, a fibroblast, a smooth muscle cell, a vascular endothelial cell, an epithelial cell, a neural stem cell, a glial cell, an osteoblast, a chondrocyte, a liver stem cell, a hematopoietic stem cell, a stromal cell, an embryonic stem cell, and a bone marrow stem cell.

Preferably, the seed cell includes but is not limited to one or more of a muscle stem cell, a myoblast, a muscle satellite cell, and a muscle precursor cell.

The biomaterial in the hydrogel solution is one or more of collagen, recombinant collagen, gelatin, matrigel, hyaluronic acid, silk fibroin, elastin, spidroin, fibrin, fibrinogen, fibroin, laminin, fibronectin, integrin, cadherin, entactin, decellularized extracellular matrix, chondroitin sulfate, heparin, keratin sulfate, dermatan sulfate, heparan sulfate, keratin, keratin sulfate, cellulose, polymerin, carboxymethyl cellulose, polylactic acid, polyvinyl alcohol, lecithin, nanocellulose, soy protein, pea protein, gluten protein, rice protein, peanut protein, yeast protein, fungal protein, wheat protein, potato protein, corn protein, chickpea protein, mung bean protein, seaweed protein, almond protein, and quinoa protein, and other biocompatible materials capable of providing adhesion sites for seed cells.

Preferably, the biomaterial is one or more of collagen, recombinant collagen, gelatin, matrigel, hyaluronic acid, and silk fibroin.

The basic culture medium is one or more of F-10, DMEM, MEM, F-12, DMEM/F-12, DMEM/F-12 GlutaMAX™, F-12K, RPMI 1640, IMDM, L-15, 199, MCDB 131, LHC, and McCoy's 5A.

Preferably, the basic culture medium is one or more of F-10, DMEM, MEM, F-12, and DMEM/F-12.

The cross-linking agent includes but is not limited to one or more of NaOH, KOH, NaHCO₃, an HEPES balanced salt solution, an EBSS balanced salt solution, an HBSS balanced salt solution, PBS, DPBS, a transglutaminase, a tyrosinase, a laccase, a lysyl oxidase, a polyphenol oxidase, a catalase, a thrombin, genipin, and other chemical cross-linking agent.

Preferably, the cross-linking agent includes one or more of NaOH, KOH, and NaHCO₃.

Preferably, the hydrogel solution includes a biomaterial, a cross-linking agent, and a basic culture medium containing a calcium salt and a seed cell; each 1 mL of the hydrogel solution contains 290-699 μL of 4-8 mg/mL of the biomaterial, 1-10 μL of 1-2 mol/L of the cross-linking agent, and 300-700 μL of the basic culture medium containing 15-25 mg/mL of the calcium salt and 1×10⁷-1×10⁸ seed cells; the biomaterial is one or more of collagen, recombinant collagen, gelatin, matrigel, hyaluronic acid, and silk fibroin; the cross-linking agent is one or more of NaOH, KOH, and NaHCO₃; the calcium salt is one or more of calcium chloride, calcium carbonate, calcium sulfate, and calcium nitrate; the basic culture medium is one or more of F-10, DMEM, MEM, F-12, and DMEM/F-12; and the seed cell is one or more of a muscle stem cell, a myoblast, a muscle satellite cell, and a muscle precursor cell of a pig, cattle, sheep, a chicken, and a duck.

More preferably, the hydrogel solution includes a biomaterial, a cross-linking agent, and a basic culture medium containing a calcium salt and a seed cell; and in each 1 mL of the hydrogel solution, the biomaterial is 150-650 μL of 6 mg/mL of collagen and 40-149 μL of matrigel, the cross-linking agent is 1-10 μL of 1 mol/L of an alkaline solution, and the basic culture medium containing a calcium salt and seed cells is resuspending 1.5×10⁶-1.5×10⁸ muscle stem cells by 300-700 μL of a DMEM solution containing 15-25 mg/mL of the calcium salt.

Preferably, the seed cell is a porcine muscle stem cell; the hydrogel solution includes a biomaterial, a cross-linking agent, and a basic culture medium containing a calcium salt and a seed cell; and in each 1 mL of the hydrogel solution, the biomaterial is 600 L of 6 mg/mL of collagen and 97 μL of matrigel, the cross-linking agent is 3 μL of 1 mol/L of a NaOH solution, and the basic culture medium containing a calcium salt and seed cells is resuspending 1.5×10⁷ porcine muscle stem cells by 300 μL of a DMEM solution containing 20 mg/mL of calcium chloride.

The method for preparing the microfluidic biomimetic fiber for cultured meat production of the present invention includes the following steps:
(1) preparing microfluidic inner and outer phase fluids: preparing the polymer solution with cell non-adhesion as an outer phase fluid and preparing the hydrogel solution containing a seed cell as an inner phase fluid; and
(2) preparing a biomimetic fiber: respectively introducing the inner and outer phase fluids prepared in step (1) into inner and outer phase channels of a microfluidic device, enabling the two phase fluids in the channels of the microfluidic device to form a stable laminar flow structure by adjusting the flow velocities of the inner and outer phases, extruding same by the microfluidic device, and then treating same by a collection medium to obtain the biomimetic fiber.

A material for manufacturing the microfluidic device in step (2) includes but is not limited to one or more of crystalline silicon, polydimethoxysiloxane, glass, quartz, polyphthalamide, polymethyl methacrylate, polycarbonate, polystyrene, epoxy resin, acrylic acid, rubber, and fluoroplastic.

A channel structure of the microfluidic device in step (2) is may be a simple form that an inner phase channel and an outer phase channel are coaxially nested; or a coaxially nested form constructed by a collection phase channel and an observation phase channel on the basis of the coaxial nesting of the inner phase channel and the outer phase channel.

Preferably, the size of a pipe diameter of the inner phase channel in the microfluidic device in step (2) ranges from 50 μm to 300 μm, and the size of a pipe diameter of an outlet of the outer phase channel ranges from 200 μm to 800 μm.

Further, in step (2), the flow velocity of an inner phase solution ranges from 0.5 mL/h to 10 mL/h, and the flow velocity of the outer phase solution ranges from 0.5 mL/h to 10 mL/h.

In step (2), the two phase fluids in the channels of the microfluidic device are enabled to form a stable laminar flow structure by adjusting the flow velocities of the inner and outer phases and extruded into the collection medium, and the biomimetic fiber is obtained after the residual collecting liquid is washed away; or in step (2), the two phase fluids in the channels of the microfluidic device are enabled to form a stable laminar flow structure by adjusting the flow velocities of the inner and outer phases, and the extruded biomimetic fiber is directly organizationally integrated and then soaked in the collection medium to form a biomimetic fiber three-dimensional tissue.

Further, the collection medium in step (2) includes but is not limited to one or more of a calcium salt solution, a sodium salt solution, a potassium salt solution, and a magnesium salt solution.

During the specific preparation, the inner and outer phase fluids are respectively filled into an syringe, then polyethylene tubes are used for connecting outlets of the syringes and inlets of the inner and outer phase channels of the microfluidic device, and then the syringes are fixed on peristaltic pumps; the peristaltic pumps push pistons of the syringes, the inner and outer phase fluids flow into the microfluidic device through the polyethylene tubes and are directly formed into the biomimetic fiber at an outlet of the device for subsequent organizational integration.

Preferably, when the biomimetic fiber is prepared, the outlet of the microfluidic device is extended into a culture dish filled with a collection medium, the two phase fluids are directly extruded into the collection medium through the outlet of the device for cross-linking and forming, the shape and the structure of the biomimetic fiber are further stabilized in the collection medium, and a temporary storage container can be provided for the biomimetic fiber before the biomimetic fiber is transferred to a cleaning fluid and a culture medium to facilitate a subsequent production operation. When the biomimetic fiber is directly organizationally integrated, the collection medium is not used in the organizational integration process, and the collection medium is used for integral cross-linking and shape fixing of the three-dimensional tissue after the forming.

Preferably, the biomimetic fiber can be washed in a washing medium to sufficiently remove the residual collection medium. The washing medium includes but is not limited to a serum-containing culture medium, a basic culture medium, phosphate buffered saline (PBS), normal saline, a glucose solution, and sterile water.

Use of the microfluidic biomimetic fiber prepared by the preparation method of the present invention in cultured meat production.

The cultured meat production includes the following steps:
transferring the biomimetic fiber into a proliferation culture medium for proliferation culture and replacing the proliferation culture medium with a differentiation culture medium after the seed cells are spontaneously fused in the biomimetic fiber to form a fiber structure; and collecting the biomimetic fiber which is proliferated, differentiated and cultured to be a mature biomimetic fiber for producing cultured meat after organizational integration and food processing.

Preferably, the produced biomimetic fiber is directly washed, transferred to the culture dish filled with the proliferation culture medium to ensure that the biomimetic fiber is completely immersed by the culture medium, and cultured in a 5% CO₂ culture box at 37° C., and the culture medium is changed once in two days.

Further, after the biomimetic fiber is subjected to the proliferation culture for 2 days and the proliferation culture medium is replaced with the differentiation culture medium to continue a differentiation culture, that is, ½ volume of the differentiation culture medium is replaced every two days; and after 7 days of the differentiation, the biomimetic fiber is harvested.

In the present invention, in order to ensure that the seed cells are fused in the biomimetic fiber to form a fiber structure: firstly, the channel size of the microfluidic device and the use amount of cells are preferably selected to tightly arrange the cells generated in a fiber inner core; secondly, the produced biomimetic fiber outer shell has the non-adhesiveness and cells are constrained in the inner core to grow; and thirdly, under the conditions of spatial constraints and tight arrangement, cells have physiological properties that tend to fuse with each other during culture to form a whole.

The proliferation culture medium includes 79%-89% by volume of a basic culture medium, 10%-20% by volume of fetal bovine serum, and 1% by volume of penicillin-streptomycin, and then 1-10 ng/ml of a basic fibroblast growth factor (bFGF) is added to the solution.

The differentiation culture medium includes 94%-97% by volume of a basic culture medium, 2%-5% by volume of horse serum, and 1% by volume of penicillin-streptomycin.

Preferably, the basic culture medium in the proliferation culture medium and the differentiation culture medium includes but is not limited to F-10, DMEM, MEM, F-12, DMEM/F-12, DMEM/F-12 GlutaMAX™, F-12K, RPMI 1640, IMDM, L-15, 199, MCDB 131, LHC, and McCoy's 5A.

Further, a polymer part of the microfluidic biomimetic fiber can be removed to obtain a pure cell fiber. A specific method is that one or more of an alginate lyase, sodium citrate, ethylene diamine tetraacetic acid, a chitosanase, a pectinase, and a carrageenase are used.

The organizational integration includes but is not limited to stacking, weaving, winding, binding, or folding.

The food processing includes pretreatment and cooking, wherein the pretreatment includes washing, seasoning, color enhancement, shaping, or sensory quality modification, and the cooking includes decocting, frying, boiling, steaming, or baking.

Further, a polymer part of the mature biomimetic fiber can be removed to obtain a pure cell fiber, a lysis solution is used in the removing method, and the lysis solution includes an alginate lyase, sodium citrate, ethylene diamine tetraacetic acid, a chitosanase, a pectinase, or a carrageenase. The outer shell of the biomimetic fiber of the present invention is removed after the biomimetic fiber is differentiated and mature for subsequent processing, and a pure cell fiber which is higher in relative protein content and richer in nutrition is obtained.

The present invention utilizes a structural bionic principle of a basic unit of a natural skeletal muscle tissue, muscle fiber, and a proper fiber carrier is given to the seed cells to ensure a very good effect in high-efficiency production of cultured meat; and the microfluidic technology is used in the cultured meat production for the first time, and a trace amount of fluids can be controlled in the micro-sized channels.

The muscle fiber is the most basic component unit of the skeletal muscle tissue and numerous muscle fibers are wrapped by connective tissue film layers to form a large piece of the skeletal muscle tissue. Inspired by a fibrous structure of the basic unit, namely the muscle fiber in the natural skeletal muscle tissue, the present invention provides a method for preparing a biomimetic fiber carrier for cultured meat production based on a microfluidic technology by taking the muscle fiber bionic as a design principle. Besides, no relevant report is found in the art for producing cultured meat by using the method. The present invention prepares the biomimetic fiber based on the microfluidic technology and the biomimetic fiber is used for producing cultured meat. The preparation process of the fiber is continuous and rapid, the prepared fiber has a good bionic property, and the directional growth capability and differentiation capability of seed cells growing in the fiber are greatly improved. The present invention prepares the biomimetic fiber having a shell-core structure based on the microfluidic technology, the seed cells are wrapped in the polymer outer shell with cell non-adhesion and show highly directional and fused growth characteristics under the spatial constraint of the outer shell, the in-vitro myogenic differentiation capability is also significantly improved, and the production efficiency is improved. Besides, the prepared fiber is very similar to the natural skeletal muscle fiber in shapes and physiological characteristics, such that the fiber prepared based on the microfluidic technology has a better bionic property.

Specifically, the preparation of the microfluidic biomimetic fiber is emphasized in the present invention. A coaxially nested microfluidic device is firstly designed and built in the present invention; then inner and outer phase fluid materials are prepared, and the biomimetic fiber is stably produced by adjusting parameters such as the introducing sequence, the flow velocity and the like of the inner and outer phase fluids, and cultured; and finally, the biomimetic fiber obtained by the culture is subjected to organizational integration and food processing to obtain a cultured meat product. Compared with the common production modes of using a bracket, a blocky hydrogel carrier and the like, the seed cells are wrapped in the polymer outer shell of the biomimetic fiber with cell non-adhesion and show highly directional and fused growth characteristics in the hydrogel inner core under the spatial constraint of the outer shell, the in-vitro myogenic differentiation capability is also significantly improved, and the production efficiency is improved.

Through the specific shell-core structure, blending of inner and outer phase components, and the whole solution design, the present invention effectively improves the differentiation capability of primary livestock and poultry cells such as muscle stem cells in a microfiber, the synthesis of muscle-related proteins is increased, mature muscle fibers are further formed, and the production efficiency of cultured meat is improved. The microfiber prepared by the present invention shows spontaneous contraction and pulsation after the proliferation culture for 2 days and the differentiation culture for 14 days, that is primary myoblasts migrate and are fused in a specific inner core wrapped by a calcium alginate outer shell to form multinucleated myotubes, and the multinucleated myotubes are further differentiated and highly express myosin, such that mature muscle fibers are formed and show certain physiological functions.

Beneficial effects: compared with the prior art, the present invention has the following advantages:
(1) the present invention prepares the continuous and large-scale biomimetic fiber with a uniform structure and a controllable size based on the microfluidic technology, the related equipment has a low cost, the preparation condition is mild, the operation is simple, and the forming is rapid;
(2) the present invention is inspired by the muscle fiber structure in the natural skeletal muscle tissue, and thus the prepared biomimetic fiber can provide a good fiber carrier for seed cells, further simulates the three-dimensional growth environment of the seed cells in vivo, and has a better bionic property;
(3) the biomimetic fiber outer shell prepared by the present invention does not have cell adhesiveness and thus can induce the seed cells to be directionally arranged, migrate, and be grown in a fused manner in an inner core space of a fiber carrier, such that the differentiation capacity of the seed cells is significantly improved, the synthesis of muscle-related proteins is increased, and the production efficiency of the cultured meat is improved; and
(4) the biomimetic fiber prepared by the present invention has the good organizational integration characteristic of the fiber material, can be further used for fine and deep processing operations such as weaving, winding, stacking and the like, and realizes the construction of large cultured meat.

### BRIEF DESCRIPTION OF THE DRAWINGS

FIG. 1 is a schematic diagram of the preparation of the microfluidic biomimetic fiber for cultured meat production according to the present invention;

FIG. 2 shows a real-time image of the preparation process of the microfluidic biomimetic fiber for cultured meat production and a structure diagram of a microfluidic device channel according to the present invention;

FIG. 3 is a feasibility verification data diagram of a flexible adjustment of the size of the microfluidic biomimetic fiber for cultured meat production, wherein (a) is light field diagrams of the biomimetic fiber with different sizes of inner cores, and the scale bar is 200 μm; and (b) and (c) show changes of the sizes of the outer shell and the inner core with changes of the flow velocities;

FIG. 4 is a structural schematic diagram and a photomicrograph of the microfluidic device channel for organizational integration according to the present invention with the scale bar of 200 μm;

FIG. 5 shows microscope bright field view diagrams of the culture process of the microfluidic biomimetic fiber for cultured meat production, wherein (a) is the microscope bright field view diagram of the biomimetic fiber incubated for 2 hours after the preparation, (b) is the microscope bright field view diagram of the biomimetic fiber after being subjected to a proliferation culture for 2 days, (c) is the microscope bright field view diagram of the biomimetic fiber after being subjected to a differentiation culture for 3 days, and (d) is the microscope bright field view diagram of the biomimetic fiber after being subjected to the differentiation culture for 7 days with the scale bar of 200 μm;

FIG. 6 shows data diagrams of changes of expression levels of cell differentiation-related genes and proteins based on qPCR and Western Blot during the differentiation culture process of the microfluidic biomimetic fiber for cultured meat production, wherein (a) is a MyoG gene, (b) is a MyHC-2a gene, (c) is a MyHC-slow gene, (d) is a Western Blot band diagram of related proteins, (e) is an analysis of band gray values of a MyoG protein, and (f) is an analysis of band gray values of a Myosin protein;

FIG. 7 shows immunofluorescence staining maps and statistical diagrams of the microfluidic biomimetic fiber for cultured meat production cultured to be mature, wherein (a) is the immunofluorescence staining map, i shows cell nuclei, ii shows a cytoskeletal protein, iii shows myosin, and iv is a fusion image, and the scale bar is 100 μm, (b) is the statistical analysis diagram of the orientation of the cytoskeletal protein, (c) is the statistical analysis diagram of circularity and aspect ratio of cell nuclei, and (d) is the statistical analysis diagrams of myosin positive cells and myotube area;

FIG. 8 shows comparison of electron microscope images of the microfluidic biomimetic fiber for cultured meat production cultured to be mature and commercial pork, wherein (a) is the cultured mature biomimetic fiber and the scale bar is 40 μm; and (b) is the commercial pork with the scale bar of 100 μm;

FIG. 9 shows comparison images of H&E staining of the microfluidic biomimetic fiber for cultured meat production cultured to be mature and the commercial pork, wherein (a) is the longitudinal cutting image of the cultured mature biomimetic fiber, (b) is the longitudinal cutting image of the commercial pork, (c) is the transverse cutting image of the cultured mature biomimetic fiber, and (d) is the transverse cutting image of the commercial pork with the scale bar of 100 μm;

FIG. 10 is a schematic diagram of a device for organizational integration of the microfluidic biomimetic fiber for cultured meat production;

FIG. 11 shows the organizational integration process (a) and the finished product (b) of the microfluidic biomimetic fiber for cultured meat production with the scale bar of 1,000 μm;

FIG. 12 is a data map comparing the content of various amino acids of the microfluidic biomimetic fiber for cultured meat production cultured to be matured with a control group and the commercial pork;

FIG. 13 shows images of polyacrylamide gel electrophoresis of the protein composition of the produced cultured meat with commercial pork; and

FIG. 14 shows data maps of comparison of texture characteristics of the finished product obtained by subjecting the produced cultured meat to the food processing and the commercial pork, wherein (a) is hardness, (b) is chewiness, (c) is elasticity, and (d) is cohesiveness.

### DESCRIPTION OF THE EMBODIMENTS

The present invention will be further described with reference to the accompanying drawings and the examples below.

The raw materials and reagents used in the examples are all commercially available. Seed cells are all obtained by a conventional separation and purification method or directly obtained commercially.

### Example 1

### Preparation of Microfluidic Inner and Outer Phase Fluids

### (1) Preparation of Outer Phase Fluid

A proper amount of sodium alginate powder was taken, placed in a super clean bench, sterilized by ultraviolet irradiation, and stood overnight. 20 mL of sterile water was measured by using a pipette and put into a centrifuge tube, 0.6 g of sodium alginate powder was weighed by using an electronic balance in a super clean bench and poured into the centrifuge tube to be uniformly mixed by using a vortex mixer, then the centrifuge tube was put into a 37° C. constant-temperature water bath kettle to be incubated for 15 min and taken out to be vortexed again, the operation was repeated for 3-5 times until the sodium alginate powder was completely dissolved to prepare a 30 mg/mL sodium alginate solution, and bubbles in the sodium alginate solution were removed by a centrifugation at 3,000×g for 5 min for later use.

### (2) Preparation of Inner Phase Fluid

0.1 g of calcium chloride was weighed into a centrifuge tube and dissolved by 5 mL of a phenol red-containing DMEM basic culture medium (C11995500CP, Gibco) to prepare a DMEM solution containing 20 mg/mL of calcium chloride, and the solution was sterilized by filtration by using a 0.22-μm filter membrane and stored on ice for later use; and 0.2 g of NaOH was weighed in a centrifuge tube and dissolved by 5 mL of ultrapure water to prepare 1 mol/L of a NaOH solution, and the solution was sterilized by filtration by using a 0.22-μm filter membrane and stored on ice for later use.

Taking 1 mL of the inner phase fluid system as an example, a cell suspension containing 1.5×10⁷ porcine muscle stem cells was placed in a centrifuge tube and centrifuged for 5 min at 300×g, a supernatant was removed, and a cell precipitate was placed on ice for storage for later use. 1.5×10⁷ porcine muscle stem cells were resuspended in 300 μL of the DMEM solution containing 20 mg/mL of calcium chloride, 600 μL of 6 mg/mL collagen (from cow hide, Sigma, type C2124) was added to the cell suspension, the whole was transferred to a 2-mL centrifuge tube containing 3 μL of 1 mol/L of a NaOH solution, then 97 μL of matrigel (standard Matrigel, Corning reagent company) was added, the mixture was lightly beaten and mixed uniformly by a 1-mL pipette tip, and finally an obtained hydrogel solution was placed on ice for storage for later use.

### Example 2

### Preparation of Microfluidic Biomimetic Fiber

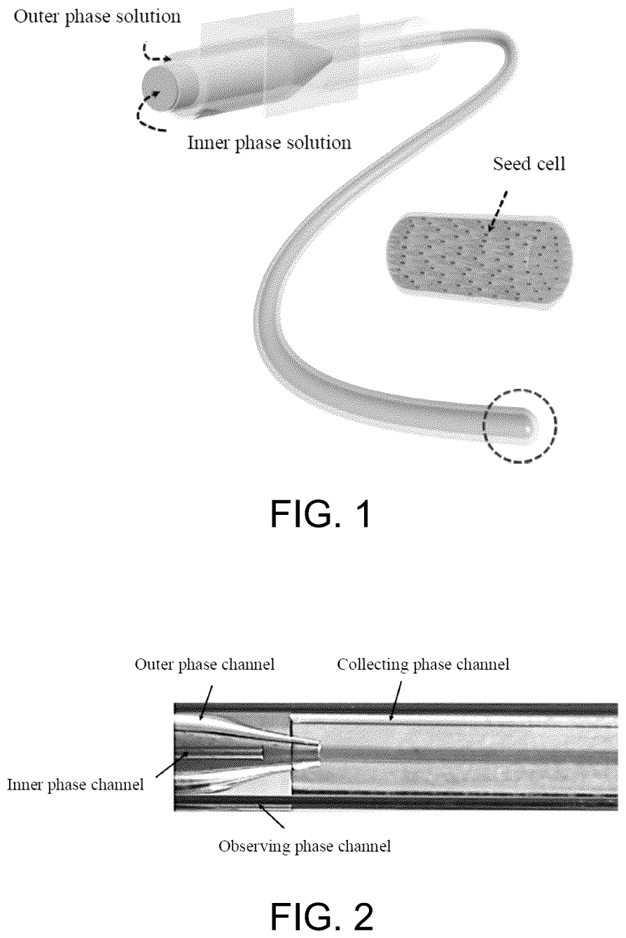

A preparation process of a microfluidic biomimetic fiber was shown in FIG. 1. A cylindrical glass capillary tube with the inner diameter of 580 μm and the outer diameter of 1,000 um was selected, and an outlet was drawn into the inner diameter of about 80 μm to serve as an inner phase channel; and then a cylindrical glass capillary tube with the inner diameter of 580 μm and the outer diameter of 1,000 μm was selected, and an outlet was drawn into the inner diameter of about 200 μm to serve as an outer phase channel. In addition, a round glass capillary tube with the inner diameter of 0.8 mm and the outer diameter of 1 mm was selected as a collection phase channel; and a square glass capillary tube with the inner edge length of 1.05 mm was selected as an observation phase channel. The observation phase square tube channel was fixed at a right middle position of a glass slide plane (the thickness of the glass slide was 1 mm; and the length of the glass slide was 30 mm and the width was 25 mm), the formation of fibers in the channel was observed by being connected to a CCD camera, then a drawing end of the outer phase channel and the collection phase channel were respectively inserted into two sections of the square tube channel, ensuring that the outer phase channel was inserted into the collection phase channel and the two channels were not blocked with each other, the outer phase channel and the collection phase channel were adjusted to the same axis (a transverse axis parallel to the length of the glass capillary tube) under a stereo microscope, and the two tubes were fixed; and then a drawing end of the inner phase channel was inserted into the outer phase channel from one side of the outer phase channel and fixed, ensuring that the two channels were not blocked with each other, and the positions of the four glass capillary tubes were adjusted to ensure that the axes (the transverse axis parallel to the length of the glass capillary tube) thereof were overlapped. Finally, 20 G dispensing needles were fixed at joints of all the channels and the microfluidic device with a coaxial nesting form was assembled after the needles were adhered by an AB glue. The structure of the microfluidic device was shown in FIG. 2.

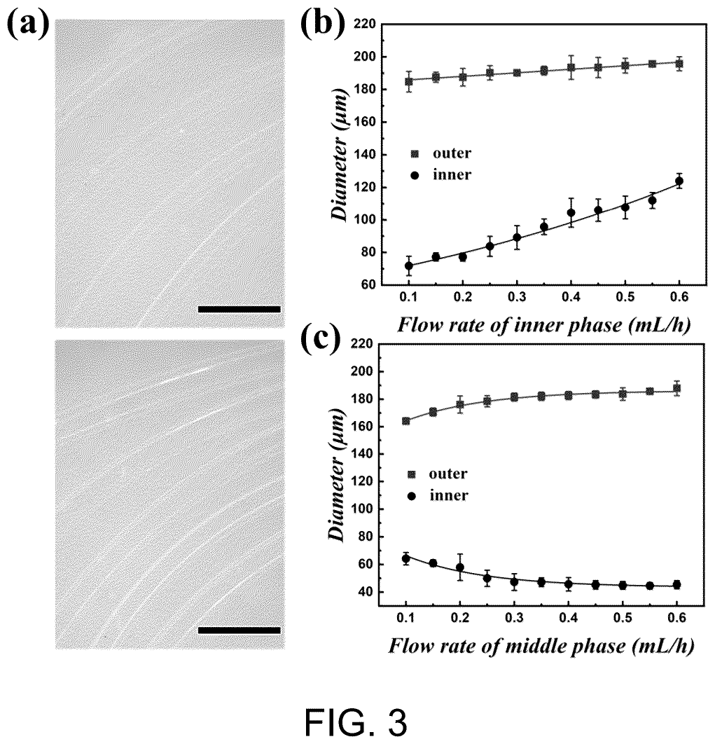

A 10 mg/mL calcium chloride solution was prepared, sterilized, and used as a collection medium. The collection medium was added into an syringe, one end of a section of a polyethylene tube was connected with a needle of the syringe, and one end of the polyethylene tube was connected with an inlet of the collection phase channel of the microfluidic device; the sodium alginate solution prepared in example 1 was added into the syringe, one end of a section of a polyethylene tube was connected with a needle of the syringe, and one end of the polyethylene tube was connected with an inlet of the outer phase channel of the microfluidic device; and the hydrogel solution containing porcine muscle stem cells prepared in example 1 was added into the syringe, one end of a polyethylene tube was connected with a needle of the syringe, and one end of the polyethylene tube was connected with an inlet of the inner phase channel of the microfluidic device. Then the syringes filled with each phase fluid were respectively fixed on a peristaltic pump, the flow velocity of the collection phase calcium chloride solution was adjusted to be 15 mL/h, the flow velocity of the inner phase hydrogel solution was 1.8 mL/h, the flow velocity of the outer phase sodium alginate solution was 2 mL/h, and the peristaltic pump was started. The production process of the shell-core type biomimetic fiber in the microfluidic device can be divided into two stages. In the first stage, the inner phase fluid and the outer phase fluid were firstly converged between an outlet of the inner phase channel and an outlet of the outer phase channel to form coaxial laminar flow fluids, and then the fluid entered the collection phase channel and the fluid and a collection phase solution were converged again to form three layers of coaxial laminar flow fluids; and in the second stage, after the three layers of fluids were formed, the outer phase sodium alginate solution started to form a calcium alginate hydrogel in the presence of calcium ions in the collection phase and inner phase solutions and continuously diffused towards the inner layer, and the shell-core type biomimetic fiber was continuously solidified and extruded into the collection medium. The three phase fluids contacted in the device and formed a stable laminar structure (FIG. 2, obvious boundaries among the collection phase fluid, the inner phase fluid, and the outer phase fluid were observed microscopically, that is a laminar flow structure phenomenon), and then the three phase fluids were extruded into the collection medium through the collection phase channel so as to obtain the biomimetic fiber. The prepared biomimetic fiber had the controllable size, was continuous and large-scale, and can be flexibly regulated and controlled by changing the flow velocity (FIG. 3). In FIG. 3, (a) was light field diagrams of the biomimetic fiber with different sizes of inner cores. As shown in (b) of FIG. 3, with the flow velocity of the inner phase solution increased, the inner phase diameter of the shell-core biomimetic fiber also increased, while the outer phase diameter slightly increased, which was less affected by the flow velocity of the inner phase solution; and as shown in (c) of FIG. 3, with the flow velocity of the outer phase solution increased, the inner phase diameter of the shell-core biomimetic fiber decreased, while the outer phase diameter also slightly increased, which was less affected by the flow velocity of the outer phase solution. It can be concluded that the inner phase diameter of the shell-core biomimetic fiber was affected by the flow velocity of each phase and in direct proportion to the flow velocity of the inner phase solution and in inverse proportion to the flow velocity of the outer phase solution; and the outer phase diameter of the biomimetic fiber was hardly affected by the flow velocity of each phase and limited by the caliber of the outlet of the microfluidic device.

In the present example, the biomimetic fiber can be rapidly and continuously produced from the outlet of the microfluidic device under the condition of ensuring that the inner and outer phase fluids were sufficient; the size of the prepared biomimetic fiber can be controlled by simply adjusting the caliber of the outlet of the microfluidic device and the flow velocities of the inner and outer phases; and the glass capillary tubes, the glass slides, the dispensing needles and the like used for building the microfluidic device were all common consumables with a low cost. In addition, during the preparation, the outer phase solution was firstly introduced and then the inner phase solution was introduced. If the order was reversed, the fiber cannot be formed.

### Example 3

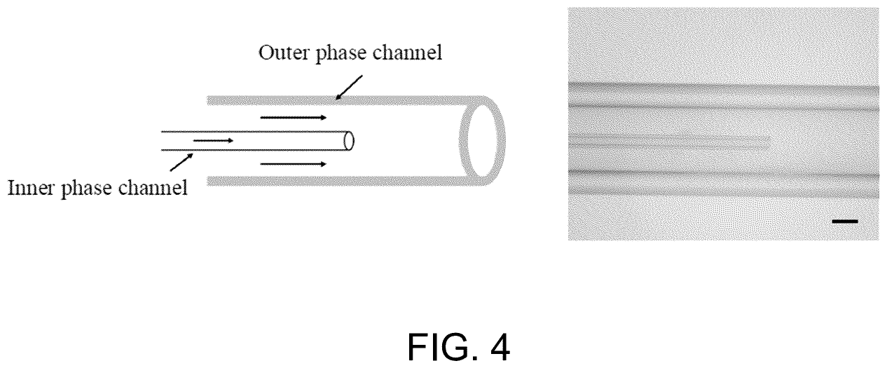

A cylindrical glass capillary tube with the inner diameter of 580 μm and the outer diameter of 1,000 μm was selected, and an outlet was drawn into the inner diameter of about 80 μm to serve as an inner phase channel; and then a cylindrical glass capillary tube with the inner diameter of 580 μm and the outer diameter of 1,000 μm was selected, and an outlet was drawn into the inner diameter of about 200 μm to serve as an outer phase channel. The outer phase channel was fixed at a right middle position of a glass slide, then a drawing end of the inner phase channel was inserted from one end of the outer phase channel, ensuring that the two phase channels were not blocked with each other, the outer phase channel and the inner phase channel were adjusted to be on the same axis under a stereo microscope, and the two tubes were fixed; and then 20 G dispensing needles were fixed at joints of the two phase channels, the assembly was completed after the two phase channels were adhered by an AB glue, the structural schematic diagram and the photomicrograph thereof were shown in FIG. 4 and the assembled microfluidic device was used for printing a three-dimensional tissue in example 5.

The microfluidic device built in the present example was a simple version of the microfluidic device built in example 2, did not contain a collection phase channel and an observation phase channel, was in a form of coaxially nesting the inner phase channel and the outer phase channel, and can be directly used for organizational integration of a biomimetic fiber. The microfluidic device built in example 2 was mainly used for observing a fluid state in the channel and real-time observation of the fiber production process during the preparation of the biomimetic fiber. In addition, the biomimetic fibers prepared on the basis of the two microfluidic devices had no difference in the structure, form and functions.

### Example 4

### Culture of Microfluidic Biomimetic Fiber

20 mL of an F-10 basic culture medium was added to a 10-cm sterile cell culture dish as a washing medium, and 3 biomimetic fibers of about 20 cm prepared in example 2 were clamped at one end by elbow forceps and washed 2-3 times in the washing medium to remove the residual collection medium sufficiently. After the washing, the biomimetic fibers were transferred to a 10-cm sterile cell culture dish containing a proliferation culture medium (84% by volume of F-10 (Gibco, 11550043), 15% by volume of fetal bovine serum (Gibco, 10270-106), and 1% by volume of penicillin-streptomycin (Gibco, 15140122), and a fibroblast growth factor bFGF (R&D, 233-FB-500/CF) with the final concentration of 5 ng/mL), and then the dish was placed in a 5% CO₂ culture box at 37° C. for a proliferation culture for 2 days. The biomimetic fibers were observed under a microscope bright field view. When porcine muscle stem cells in the biomimetic fibers fully migrated and were fused to form a fibrous structure, the proliferation culture medium was discarded by sucking, and then the biomimetic fibers were washed 2-3 times by using a serum-free DMEM basic culture medium. After the washing, a differentiation culture medium (97% by volume of DMEM (C11995500CP, Gibco), 2% by volume of horse serum (Hyclone, SH30074.02), and 1% by volume of penicillin-streptomycin (Gibco, 15140122)) was added into the culture dish, the culture dish was placed at 37° C. and 5% CO₂ to continue differentiation culture, then ½ of the differentiation culture medium in the culture dish was replaced every two days, and mature biomimetic fibers were obtained after the differentiation culture for 7 days.

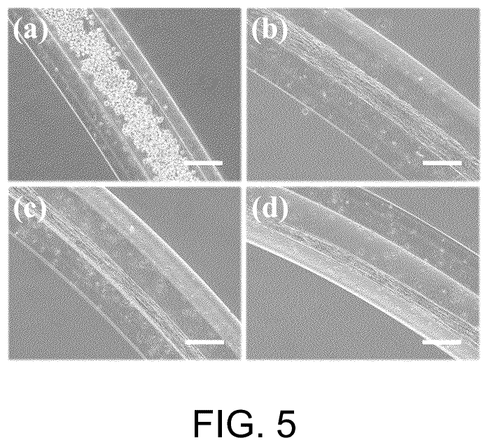

As shown in FIG. 5, after being cultured for 2 hours, seed cells remained spherical and tightly arranged in an inner core of the shell-core biomimetic fiber ((a) of FIG. 5); after the proliferation culture for 2 days, the seed cells completed migration in the inner core and fused with each other to form a fibrous structure ((b) of FIG. 5); after the differentiation culture for 3 days, the cell fiber became thinner compared to that in the proliferation culture for 2 days, and myotube structures in the cell fiber were seen, indicating that the seed cells gradually started to differentiate ((c) of FIG. 5); and after the differentiation culture for 7 days, the cell fiber was further thinned compared to that in the differentiation culture for 3 days, the myotubes in the cell fiber became longer, and the seed cells differentiated and matured ((d) of FIG. 5). The fusion of the seed cells to form a fibrous structure which was wrapped in a transparent polymer outer shell was observed in FIG. 5.

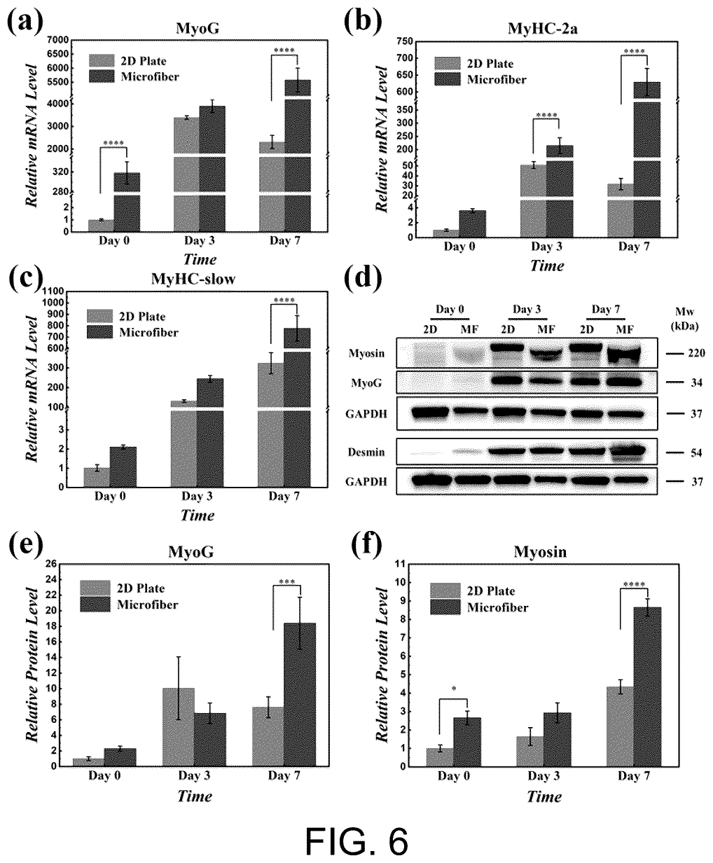

The changes of expressions of differentiation-related genes and proteins of the seed cells growing on the biomimetic fiber and a two-dimensional plate were respectively evaluated from a molecular biology level on day 0, day 3 and day 7 of the differentiation by using RT-qPCR and Western Blot, wherein the seed cells on the two-dimensional plate were prepared by directly performing differentiation culture on porcine muscle stem cells in a conventional manner, that is, the porcine muscle stem cells were inoculated onto a 3.5-cm sterile culture dish paved with matrigel for proliferation and differentiation culture, and the use amount of cells, proliferation and differentiation culture time and the like were completely consistent with those of the biomimetic fiber. On day 0, day 3 and day 7 of the differentiation, the cells in the biomimetic fiber and the two-dimensional plate were lysed using Trizol, and RNA in the lysed cells was extracted using a total RNA extraction kit for cultured cells of Tiangen Biochemical Co., Ltd.; after the concentration of RNA in a sample was determined, the RNA was subjected to reverse transcription by using a reverse transcription kit to obtain cDNA, wherein the reverse transcription program was set to be 37° C. for 15 min and 85° C. for 5 s; and then, the cDNA obtained by the reverse transcription was subjected to a qPCR reaction using a RT-qPCR kit, the target genes were MyoG, MyHC-2a, and MyHC-slow, and the reaction program was 95° C. for 30 s, 95° C. for 5 s, and 60° C. for 30 s. As shown in (a)-(c) of FIG. 6, the expression of the Myogenin (MyoG) gene in the seed cells cultured on the biomimetic fiber was 300 times higher than that in the two-dimensional plate culture control at the beginning of the differentiation (Day 0); and at the final stage of the differentiation (Day 7), the expressions of muscle maturation marker-myosin synthesis-related genes MyHC-2a and MyHC-slow in the seed cells cultured in the biomimetic fiber were both obviously higher than those of the two-dimensional plate culture control group. Further, cell protein samples were obtained by using a RIPA lysate to lyse cells in the biomimetic fiber and the two-dimensional plate on ice, the collected protein samples were centrifuged at 12,000 rpm and 4° C. for 5 min, then a supernatant was collected, the protein concentration of the samples were measured by a BCA kit, the protein concentration of the samples was diluted to 1.25 mg/mL, then 5×Loading buffer of one fourth of the volume of the samples was added, and the mixture was uniformly mixed and heated at 95° C. for 5 min to denature the proteins. 20 μL of the denatured proteins were taken from each sample and subjected to an SDS-PAGE gel electrophoresis at 80 V for 30 min and 120 V for 70 min. Then, a PVDF membrane with the proper size was cut and transferred by using a rapid wet transfer, bands (MyHC: 220 kDa; MYOG: 34 kDa; and GAPDH: 36 kDa) with the corresponding molecular weights of the proteins were cut, 5% skim milk powder was used for blocking the membrane, a primary antibody was added for incubation at 4° C. overnight, and a secondary antibody was added for incubation at room temperature for 2 h; and a developing solution A and a developing solution B were mixed at a ratio of 1:1, the mixture was dripped on the bands, incubation at a dark placed was performed for 5 min, then the developing solution was sucked off, developing was performed by using an imager, pictures were taken, and an analysis of gray values of protein bands was performed by using an imageJ software. As shown in (d)-(f) of FIG. 6, the expressions of the seed cell differentiation-related proteins and genes showed the same tendency. In conclusion, when the seed cells grew in the biomimetic fiber, the expressions of the differentiation-related genes and proteins (expressions of MyoG and Myosin proteins) were significantly higher than those of the two-dimensional culture group. In the early stage (day 0) and the final stage (day 7) of the differentiation, the MyoG protein in the seed cells in the biomimetic fiber was 2.2 times and 2.4 times higher than that of the two-dimensional culture group, respectively; and in the early stage (day 0), the middle stage (day 3), and the final stage (day 7) of the differentiation, the Myosin protein in the seed cells in the biomimetic fiber was 2.66 times, 1.78 times, and 2 times higher than that of the two-dimensional culture group, respectively, which showed that the differentiation capacity of the seed cells was obviously improved, the synthesis of muscle-related protein was increased, and the production efficiency of cultured meat was improved.

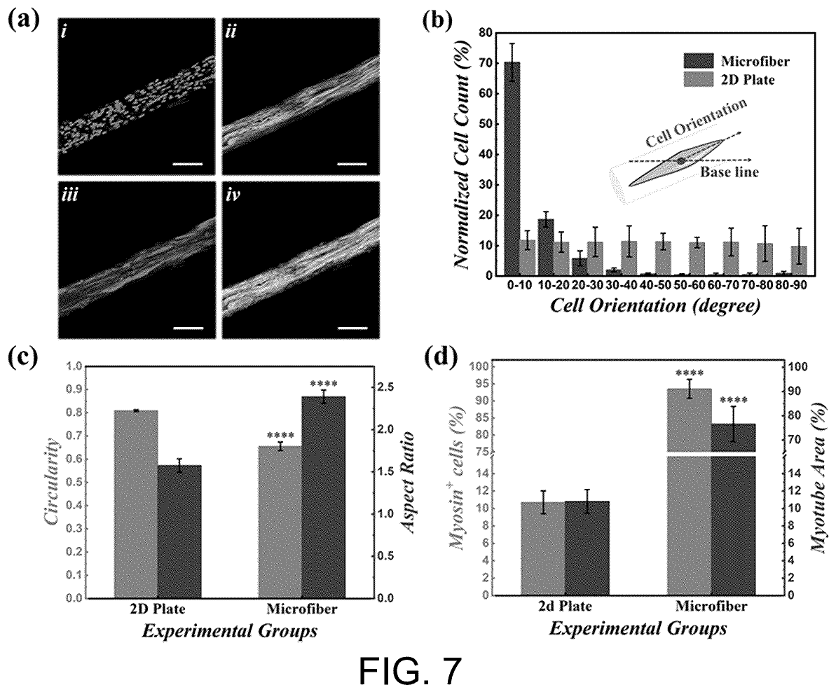

In addition, the biomimetic fiber was subjected to immunofluorescent staining, observed, and analyzed after 7 days of the differentiation. The biomimetic fibers after the 7 days of the differentiation were fixed by using 4% paraformaldehyde, the fixed sample was penetrated by using 0.5% Triton X-100 for 30 min and blocked by using a 5% BSA solution for 30 min after the penetration; a primary antibody was added for incubation at 4° C. overnight, a secondary antibody was added for incubation at room temperature for 2 h, further incubation was performed, and phalloidin was used to stain F-actin for 30 min; and finally, a mounting medium containing a DAPI cell nucleus dye was dripped on the sample for mounting, and the sample was observed and photographed by using a laser confocal microscope. As shown in (a)-(d) of FIG. 7, compared with the two-dimensional culture group, the cytoskeletal proteins in the biomimetic fiber were directionally arranged along the fiber orientation (it can be observed that the F-actin orientation was consistent with the fiber orientation, and the seed cells grew in a highly directional manner), the myogenic marker protein myosin had a higher expression, indicating that the seed cells were directionally arranged, migrated, and were grown in a fused manner in the biomimetic fiber, the differentiation capability was significantly improved, and the synthesis of the muscle-related proteins was increased.

### Example 5

### Removal of Microfluidic Biomimetic Fiber Polymer Outer Shell

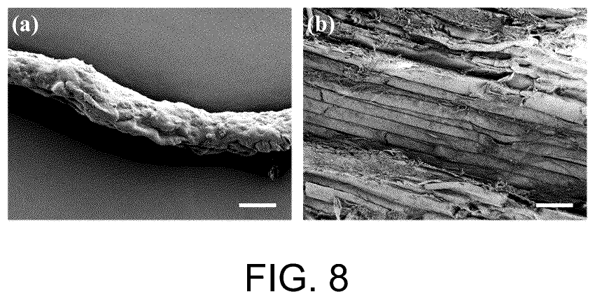

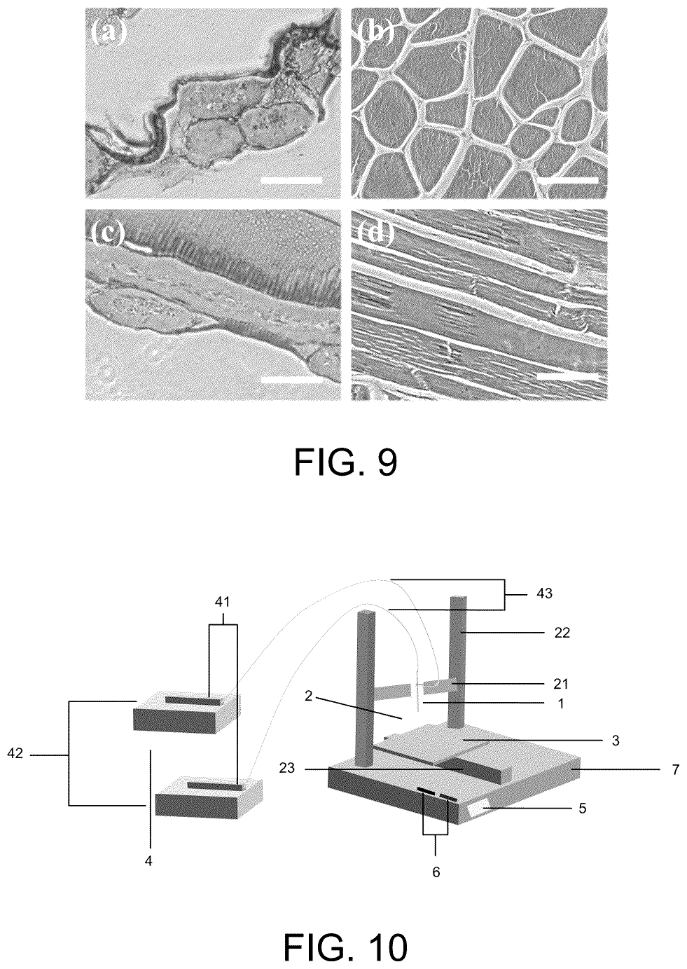

4 mg of alginate lyase dry powder was weighed and dissolved by 1 mL of ultrapure water to prepare 4 mg/mL of an alginate lyase solution, and the solution was sterilized by filtration by using a 0.22-μm filter membrane and placed in a 37° C. water bath kettle for later use. The differentiation culture medium in the culture dish of the mature biomimetic fiber obtained after 7 days of the differentiation culture in example 4 was sucked off, and the biomimetic fiber was washed 2-3 times with a serum-free DMEM basic culture medium. After the washing, 10 mL of the serum-free DMEM basic culture medium was added to the culture dish, 200 μL of an alginate lyase solution was added to the culture medium, and the culture dish was incubated in an 5% CO₂ incubator at 37° C. for 20 min. After the lysis, the cell fibers were taken out with elbow forceps and washed with PBS followed by tissue fixation with 4% of paraformaldehyde and 2.5% of glutaraldehyde. The sample fixed by 2.5% of glutaraldehyde was placed in 50%, 70%, 80%, 90% and anhydrous ethanol for gradient dehydration; the dehydrated sample was immersed into tert-butyl alcohol for replacement and then the sample was freeze-dried to remove the tert-butyl alcohol; and after the sample surface was sprayed with gold using an ion sputtering device, the sample was observed by a scanning electron microscope and photographed, and compared with commercial pork ((a) and (b) of FIG. 8). In addition, the sample fixed by 4% of paraformaldehyde was placed in 70%, 80%, 90% and anhydrous ethanol for gradient dehydration, and the ethanol in the sample was replaced by xylene in a gradient manner; the xylene in the sample was replaced with an embedding medium paraffin, and the sample was embedded with fresh paraffin, sectioned with a microtome, stained with hematoxylin and eosin staining solutions, observed by an inverted microscope and photographed, and compared with commercial pork ((a)-(d) of FIG. 9). From FIGS. 8 and 9, it can be seen that naked seed cells and myotube structures can be observed on the surface of the biomimetic fibers, and the tissue structure very similar to that of pork skeletal muscle fibers was shown.

### Example 6

### Organizational Integration of Microfluidic Biomimetic Fiber

The microfluidic device with a coaxial nesting form constructed in example 3 was integrated into a 3D printer nozzle moving system to serve as a printing nozzle, and modified to obtain a microfluidic 3D printing device. The structural schematic diagram of the device was shown in FIG. 10.

The printing device includes a printing nozzle 1, a printing moving system 2, a carrying platform 3, a sample injection system 4, a printing control display system 5, a data transmission system 6, and a base 7.

The base 7 is placed on a horizontal desktop, a y-axis movable optical axis 23 and z-axis moving optical axes 22 are fixed on the base 7 through bolts, the moving optical axes are generally made of aluminum alloy, and then an x-axis movable optical axis 21 is connected to the z-axis moving optical axe 22, namely the printing moving system 2 is successfully assembled. The printing nozzle 1 is clamped and fixed on the x-axis movable optical axis 21 in the 3D printing moving system 2 and driven by the x-axis movable optical axis 21 to move in an x-axis direction; and the x-axis movable optical axis 21 is connected with the z-axis moving optical axes 22 through bolts, and the z-axis movable optical axis 22 drives the x-axis movable optical axis to move in a z-axis direction. The carrying platform 3 is assembled on the y-axis movable optical axis 23 in the printing moving system 2 through a buckle, the carrying platform 3 and a printed product molded on the carrying platform 3 are driven to move in a y-axis direction by the y-axis movable optical axis 23, and the carrying platform 3 can be dismantled to collect a sample.

The sample injection system 4 includes sample loaders 41, sample injection pumps 42 and guide pipes 43, wherein the sample loaders 41 are fixed on the sample injection pumps 42 and can be flexibly disassembled so as to be filled with a printing material, one ends of the guide pipes 43 are connected with outlets of the sample loaders 41, the other ends of the guide pipes are connected with inlets of the printing nozzle 1, the sample injection pumps 42 are Longer Pump LSP01-1A micro-injection pumps, the sample loaders 41 can be an syringe, and the guide pipes 43 can be a polyethylene tube. The printing control display system 5, the data transmission system 6, and the base 7 are a whole, the printing control display system 5 is imbedded after a front side of the base 7 is opened, interfaces of the data transmission system 6 are imbedded after a top of the base is punched, the data transmission system 6 is connected to the printing control display system 5 through a data line, and the printing control display system 5 is connected with the printing moving system 2 through a data line. The printing control display system 5 is mainly used for controlling printing leveling, selecting a printing program, issuing a printing instruction, and adjusting the position of the printing moving system 2; the data transmission system 6 is used for transmitting a printing instruction file into a 3D printer; and the data transmission form of the data transmission system 6 includes USB transmission, memory card transmission or computer transmission.

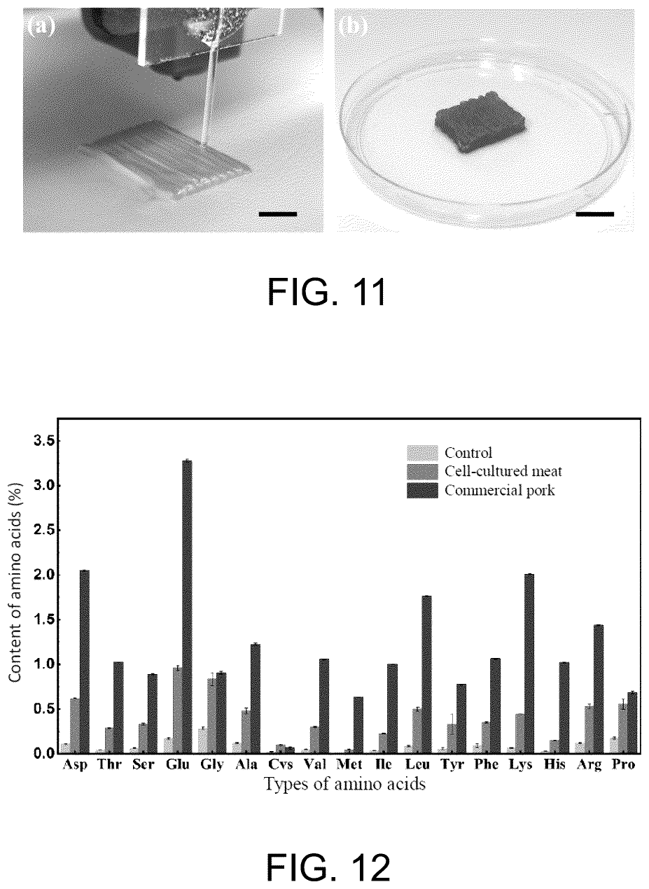

A printing model is established by using an Auto CAD 2021 software and led into the printing control display system 5 of the 3D printing device for later use through the data transmission system 6. The sodium alginate solution prepared in example 1 was added into the syringe, one end of a section of a polyethylene tube was connected with a needle of the syringe, and one end of the polyethylene tube was connected with an outer phase inlet of the microfluidic device; and the hydrogel solution containing porcine muscle stem cells prepared in example 1 was added into the syringe, one end of a polyethylene tube was connected with a needle of the syringe, and one end of the polyethylene tube was connected with an inner phase inlet of the microfluidic device. Then the syringes filled with the two phase fluids were respectively fixed on two Longer Pump LSP01-1A micro-injection pumps, and the flow velocity of the inner phase hydrogel solution was adjusted to be 1.8 mL/h, and the flow velocity of the outer phase sodium alginate solution was adjusted to be 2 mL/h. The inner and outer phase printing materials were introduced into the microfluidic device through the polyethylene tubes under the driving of the pumps. After the fibers were produced at the outlet of the device (namely, the outlet of the outer phase channel), a printing program was selected and the 3D printing device was started, then the 3D printer nozzle moving system drives the microfluidic device to move on x and z axes, the printed sample was driven to move on a y axis by the carrying platform, wherein the moving speed of each optical axis was 5 mm/s, the produced fibers were deposited on the carrying platform 3, stacked, and formed cording to a G-code printing instruction path, a three-dimensional tissue was obtained after the printing, a 10 mg/mL calcium chloride solution was prepared, sterilized, and used as a collection medium, the printed three-dimensional tissue was taken off from the carrying platform, the calcium chloride solution was slowly dripped on the three-dimensional tissue until the three-dimensional tissue was just immersed, the calcium chloride solution was suck off after cross-linking for 3 min, the organizational integration process was shown in (a) of FIG. 11, and the treated three-dimensional tissue was shown in (b) of FIG. 11. Moreover, the organizational integration may also be performed in other ways, such as stacking, weaving, winding, binding, or folding.

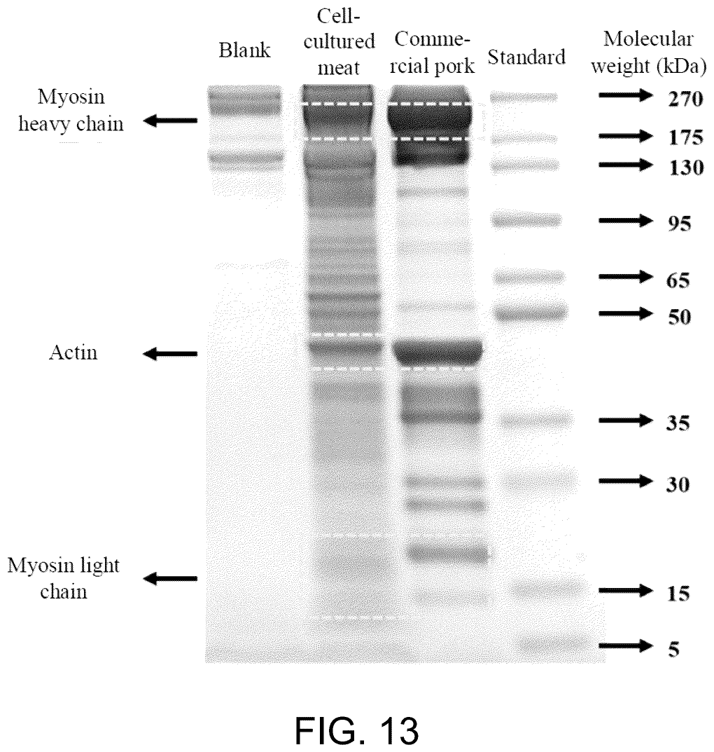

The three-dimensional tissue was transferred to a proliferation culture medium (84% by volume of DMEM/F-12, 15% by volume of fetal bovine serum, 1% by volume of penicillin-streptomycin, and 5 ng/ml of a fibroblast growth factor) for cleaning and infiltrating for 10 min, and then the three-dimensional tissue was transferred to a 5% CO₂ incubator at 37° C. for proliferation culture for 2 days; and the three-dimensional tissue was observed in a microscope bright field, the proliferation culture medium was sucked off when porcine muscle stem cells in the three-dimensional tissue sufficiently migrated and were fused to form a fibrous structure, and the three-dimensional tissue was washed with a serum-free DMEM basic culture medium for 2-3 times. After the washing, 15 mL of a differentiation culture medium (97% by volume of DMEM, 2% by volume of horse serum, and 1% by volume of penicillin-streptomycin) was added into a culture dish, the three-dimensional tissue in the culture dish was placed at 37° C. and 5% CO₂ for continuous differentiation culture, then ½ of the differentiation culture medium in the culture dish was replaced every two days, the three-dimensional tissue was subjected to food processing after 7 days of the differentiation, the three-dimensional tissue differentiated to be mature was harvested, and the three-dimensional tissue was washed with ultrapure water to remove the residual differentiation culture medium; and preliminary cultured meat was obtained and amino acid analysis results (FIG. 12) showed that the content of various amino acids of the cultured meat was obviously higher than that of a control group (an inner core hydrogel solution without seed cells was used and other preparation methods were consistent with examples 1 and 2), and the content of glycine (Gly), cysteine (Cys), and proline (Pro) was very close to that of commercial pork. The three-dimensional tissue differentiated to be mature was harvested, and the three-dimensional tissue was washed with ultrapure water to remove the residual differentiation culture medium to obtain preliminary cultured meat. As shown in gel images of an SDS-PAGE protein gel electrophoresis of FIG. 13, the types and band positions of meat-related proteins (myosin heavy chain, actin, myosin light chain protein and the like) in the cultured meat were similar to those of the commercial pork.

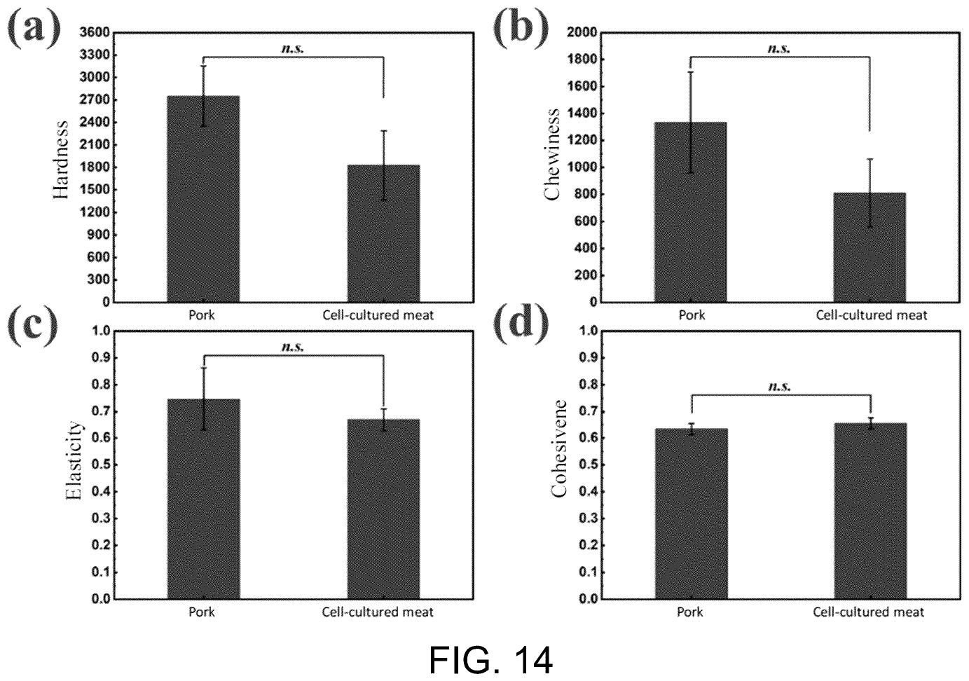

30 mg/mL of a sodium alginate solution, 50 mg/mL of a gelatin solution, 100 mg/mL of a transglutaminase solution, and 10 mg/mL of a calcium chloride solution were prepared for later use; the gelatin solution and the transglutaminase solution were mixed at a volume ratio of 9:1, the mixture was dropwise added onto the primary cultured meat to fully coat the surface of the primary cultured meat, the primary cultured meat was incubated at 37° C. for 2 h, immersed into the sodium alginate solution for 3 s, took out, and then placed in the calcium chloride solution for cross-linking for 3 min, and the residual calcium chloride solution was washed away to obtain the successfully-shaped cultured meat. The shaped cultured meat was then subjected to a food pretreatment (washing, seasoning, color enhancement, shaping, sensory quality modification and the like) and a frying treatment to obtain a cultured meat product. After the shaping treatment, a corresponding meat quality analysis was performed. The cultured meat had no significant difference from the commercial pork in 4 textural indicators of hardness, elasticity, chewiness, and cohesiveness (FIG. 14).

After the microfiber prepared by the present invention was subjected to the proliferation and differentiation culture, on the 7th day of the differentiation, comparison results by a scanning electron microscope and H&E tissue staining of the microfiber and pork showed that the microfiber had a compact structure, an obvious myotube structure can be observed on the surface, and a tissue slice also showed the staining characteristic of the microfiber was close to that of pork fibers, and the microfiber was very close to the pork fibers as a whole; and on the 14th day of the differentiation, the microfiber also showed spontaneous contraction and pulsation under a bright field observation, which was to realize crossing from cells to mature tissues under an in-vitro culture condition, indicating that the microfiber consisting of seed cells was fully matured to form a muscle fiber which had the contraction function of a natural muscle fiber. The present invention has the ability to culture a muscle fiber in vitro. By collecting and assembling these in-vitro cultured muscle fibers into a large piece of a tissue, cultured meat consisting of the muscle fibers cultured in vitro can be obtained.

### Example 7

The preparation method in example 7 was the same as that in example 1 except that the polymer solution with cell non-adhesion was chitosan with the concentration of 10 mg/mL.

The components of the hydrogel solution were 30% by volume of gelatin, 1% by volume of a genipin solution, and 69% by volume of an F-10 culture medium containing calcium sulfate and 5×10⁶ bovine muscle stem cells.

### Example 8

The preparation method in example 8 was the same as that in example 1 except that the polymer solution with cell non-adhesion was pectin with the concentration of 50 mg/mL.

The components of the hydrogel solution were 70% by volume of hyaluronic acid, 1% by volume of a carbodiimide enzyme solution, and 29% by volume of a MEM culture medium containing calcium lactate and 5×10⁸ chicken muscle stem cells.

### Example 9

The preparation method in example 9 was the same as that in example 1 except that the polymer solution with cell non-adhesion was carrageenan with the concentration of 25 mg/mL.

The components of the hydrogel solution were 50% by volume of fibrinogen, 0.5% by volume of a thrombin solution, and 49.5% by volume of a DMEM/F-12 culture medium containing calcium chloride and 5×10⁷ sheep muscle stem cells.

Under the culture condition of the shell-core structure of the specific microfluidic biomimetic fiber of the present invention, the effect of the present invention can be realized by using a certain amount of muscle stem cells of a pig, cattle, sheep, a chicken, a duck and the like for culture.

What is claimed is:

1. A microfluidic biomimetic fiber for cultured meat production, wherein the microfluidic biomimetic fiber has a shell-core structure; and an outer shell of the microfluidic biomimetic fiber is formed by cross-linking a polymer solution with cell non-adhesion, and an inner core wrapped by the outer shell is a hydrogel solution mixed with a seed cell.

2. The microfluidic biomimetic fiber for cultured meat production according to claim 1, wherein the polymer solution with cell non-adhesion is any one or more of sodium alginate, chitosan, pectin, carrageenan, and gellan gum; and the concentration of the polymer solution with cell non-adhesion is 10-50 mg/mL.

3. The microfluidic biomimetic fiber for cultured meat production according to claim 1, wherein the hydrogel solution contains 30%-70% by volume of a biomaterial, 0.01%-1% by volume of a cross-linking agent, and the balance of a basic culture medium containing a calcium salt and 5×10⁶-5×10⁸ seed cells/mL.

4. The microfluidic biomimetic fiber for cultured meat production according to claim 1, wherein the seed cell is derived from any one or more of a pig, cattle, sheep, a chicken, a duck, a rabbit, fish, a shrimp, and a crab.

5. The microfluidic biomimetic fiber for cultured meat production according to claim 1, wherein the seed cell is one or more of a muscle stem cell, a myoblast, a muscle satellite cell, a muscle precursor cell, a bone marrow-derived mesenchymal stem cell, an adipose-derived mesenchymal stem cell, an induced pluripotent stem cell, a cardiac muscle cell, an adipose stem cell, an adipose precursor cell, a bone marrow-derived adipose adult cell, a fibroblast, a smooth muscle cell, a vascular endothelial cell, an epithelial cell, a neural stem cell, a glial cell, an osteoblast, a chondrocyte, a liver stem cell, a hematopoietic stem cell, a stromal cell, an embryonic stem cell, and a bone marrow stem cell.

6. The microfluidic biomimetic fiber for cultured meat production according to claim 3, wherein the biomaterial is one or more of collagen, recombinant collagen, gelatin, matrigel, hyaluronic acid, silk fibroin, elastin, spidroin, fibrin, fibrinogen, fibroin, laminin, fibronectin, integrin, cadherin, entactin, decellularized extracellular matrix, chondroitin sulfate, heparin, keratin sulfate, dermatan sulfate, heparan sulfate, keratin, keratin sulfate, cellulose, polymerin, carboxymethyl cellulose, polylactic acid, polyvinyl alcohol, lecithin, nanocellulose, soy protein, pea protein, gluten protein, rice protein, peanut protein, yeast protein, fungal protein, wheat protein, potato protein, corn protein, chickpea protein, mung bean protein, seaweed protein, almond protein, and quinoa protein.

7. The microfluidic biomimetic fiber for cultured meat production according to claim 3, wherein the basic culture medium is one or more of F-10, DMEM, MEM, F-12, DMEM/F-12, DMEM/F-12 GlutaMAX™M, F-12K, RPMI 1640, IMDM, L-15, 199, MCDB 131, LHC, and McCoy's 5A.

8. The microfluidic biomimetic fiber for cultured meat production according to claim 3, wherein the cross-linking agent is any one or more of NaOH, KOH, NaHCO₃, an HEPES balanced salt solution, an EBSS balanced salt solution, an HBSS balanced salt solution, PBS, DPBS, a transglutaminase, a tyrosinase, a laccase, a lysyl oxidase, a polyphenol oxidase, a catalase, a thrombin, and genipin.

9. The microfluidic biomimetic fiber for cultured meat production according to claim 1, wherein the hydrogel solution comprises a biomaterial, a cross-linking agent, and a basic culture medium containing a calcium salt and a seed cell; each 1 mL of the hydrogel solution contains 290-699 μL of 4-8 mg/mL of the biomaterial, 1-10 μL of 1-2 mol/L of the cross-linking agent, and 300-700 μL of the basic culture medium containing 15-25 mg/mL of the calcium salt and 1×10⁷-1×10⁸ seed cells; the biomaterial is one or more of collagen, recombinant collagen, gelatin, matrigel, hyaluronic acid, and silk fibroin; the cross-linking agent is one or more of NaOH, KOH, and NaHCO₃; the calcium salt is one or more of calcium chloride, calcium carbonate, calcium sulfate, and calcium nitrate; the basic culture medium is one or more of F-10, DMEM, MEM, F-12, and DMEM/F-12; and the seed cell is one or more of a muscle stem cell, a myoblast, a muscle satellite cell, and a muscle precursor cell of a pig, cattle, sheep, a chicken, and a duck.

10. A method for preparing the microfluidic biomimetic fiber for cultured meat production according to claim 1, comprising the following steps:
(1) preparing microfluidic inner and outer phase fluids: preparing the polymer solution with cell non-adhesion as an outer phase fluid and preparing the hydrogel solution containing a seed cell as an inner phase fluid; and
(2) preparing a biomimetic fiber: respectively introducing the inner and outer phase fluids prepared in step (1) into inner and outer phase channels of a microfluidic device, enabling the two phase fluids in the channels of the microfluidic device to form a stable laminar flow structure by adjusting the flow velocities of the inner and outer phases, extruding same by the microfluidic device, and then treating same by a collection medium to obtain the biomimetic fiber.

11. The preparation method according to claim 10, wherein the microfluidic device in step (2) is made of one or more of crystalline silicon, polydimethoxysiloxane, glass, quartz, polyphthalamide, polymethyl methacrylate, polycarbonate, polystyrene, epoxy resin, acrylic acid, rubber, and fluoroplastic.

12. The preparation method according to claim 10, wherein a channel structure of the microfluidic device in step (2) is in a form that an inner phase channel and an outer phase channel are coaxially nested; or a coaxially nested form constructed by a collection phase channel and an observation phase channel on the basis of the coaxial nesting of the inner phase channel and the outer phase channel.

13. The preparation method according to claim 10, wherein in step (2), the two phase fluids in the channels of the microfluidic device are enabled to form a stable laminar flow structure by adjusting the flow velocities of the inner and outer phases and extruded into the collection medium, and the biomimetic fiber is obtained after the residual collecting liquid is washed away; or in step (2), the two phase fluids in the channels of the microfluidic device are enabled to form a stable laminar flow structure by adjusting the flow velocities of the inner and outer phases, and the extruded biomimetic fiber is directly organizationally integrated and then is soaked in the collection medium to form a biomimetic fiber three-dimensional tissue; and the collection medium is one or more of a calcium salt solution, a sodium salt solution, a potassium salt solution, and a magnesium salt solution.

14. Use of the microfluidic biomimetic fiber for cultured meat production according to claim 1 in the cultured meat production.

15. The use according to claim 14, wherein the cultured meat production comprises the following steps:
transferring the biomimetic fiber into a proliferation culture medium for proliferation culture and replacing the proliferation culture medium with a differentiation culture medium after the seed cells are fused in the biomimetic fiber to form a fiber structure; and collecting the biomimetic fiber which is proliferated, differentiated and cultured to be a mature biomimetic fiber for producing cultured meat after organizational integration and food processing.

16. The use according to claim 15, wherein the proliferation culture medium comprises 79%-89% by volume of a basic culture medium, 10%-20% by volume of fetal bovine serum, and 1% by volume of penicillin-streptomycin, and contains 1-10 ng/ml of a basic fibroblast growth factor; and the differentiation culture medium comprises 94%-97% by volume of a basic culture medium, 2%-5% by volume of horse serum, and 1% by volume of penicillin-streptomycin.

17. The use according to claim 15, wherein the organizational integration is performed in a manner of stacking, weaving, winding, binding, or folding.

18. The use according to claim 15, wherein the food processing comprises pretreatment and cooking, wherein the pretreatment comprises one or more of washing, seasoning, color enhancement, shaping, or sensory quality modification, and the cooking comprises decocting, frying, boiling, steaming, or baking.

19. The use according to claim 15, wherein a polymer part of the mature biomimetic fiber can be removed to obtain a pure cell fiber, a lysis solution is used in the removing method, and the lysis solution comprises an alginate lyase, sodium citrate, ethylene diamine tetraacetic acid, a chitosanase, a pectinase, or a carrageenase.
